(19) United States
(12) Patent Application Publication
(10) Pub. No.: US 2012/0150058 A1
(43) Pub. Date: Jun. 14, 2012

(54) SYSTEM AND METHOD FOR MONITORING A PATIENT FOR A RESPIRATORY ARREST/DEATH EVENT

(76) Inventor: Marc Zubrow, Wilmington, DE (US)

(21) Appl. No.: 13/312,331

(22) Filed: Dec. 6, 2011

Related U.S. Application Data
(60) Provisional application No. 61/421,597, filed on Dec. 9, 2010.
(60) Provisional application No. 61/469,793, filed on Mar. 30, 2011.

(51) Int. Cl. A61B 5/08

(52) U.S. Cl. 600/538

(57) ABSTRACT

A method for monitoring patients for a respiratory arrest/death event can include selecting a patient that is at risk of undergoing a respiratory arrest/death event, attaching a temperature sensor to the patient such that the temperature sensor is in fluid communication with air entering and exiting the patient, and processing data on a temperature of the air entering and exiting the patient collected from the temperature sensor over a continuous period of patient hospitalization.

### CLAIM FOR PRIORITY

This application claims the benefit of prior U.S. Provisional Application No. 61/421,597 filed on Dec. 9, 2010 and to U.S. Provisional Application No. 61/469,793, filed on Mar. 30, 2011, each of which is incorporated by reference in its entirety.

### TECHNICAL FIELD

This invention relates to a system and method for determining respiratory rate at a point in time and monitoring patients for a respiratory arrest/death event.

### BACKGROUND

Patients can be at risk of rapid changes in health status. Systems and methods for monitoring changes in health status can help improve the quality of care provided to patients.

### SUMMARY

In one aspect, a method for monitoring patients for a respiratory arrest/death event can include selecting a patient that is at risk of undergoing a respiratory arrest/death event, attaching a temperature sensor to the patient such that the temperature sensor is in fluid communication with air entering and exiting the patient, and processing data on a temperature of the air entering and exiting the patient collected from the temperature sensor over a continuous period during patient hospitalization. The patient can be an animal, such as a mammal, for example, a human, dog, cat, horse, cow, rat, rabbit or mouse.

In one aspect, a method for monitoring patients can include selecting a patient who has been admitted to a hospital, attaching a temperature sensor to the patient during the collection of vital sign information, such that the temperature sensor is in fluid communication with air entering and exiting the patient for a discrete time period, and processing data on a temperature of the air entering and exiting the patient collected from the temperature sensor. The discrete time period can be less than 5 minutes, less than 4 minutes, less than 3 minutes, less than 2 minutes or less than 1 minute.

In one aspect, a method for monitoring patients can include selecting a patient who is being transported by an emergency vehicle, attaching a temperature sensor to the patient during the collection of vital sign information, such that the temperature sensor is in fluid communication with air entering and exiting the patient, and processing data on a temperature of the air entering and exiting the patient collected from the temperature sensor. The processor can process data on a temperature of the air entering and exiting a patient collected from the temperature sensor over a discrete period of time. The discrete time period can be less than 5 minutes, less than 4 minutes, less than 3 minutes, less than 2 minutes or less than 1 minute.

In another aspect, a method for determining a respiratory rate of a patient can include attaching a temperature sensor to the patient, such that the temperature sensor is in fluid communication with air entering and exiting the patient, and processing data on a temperature of the air entering and exiting the patient collected from the temperature sensor. The processor can process data on a temperature of the air entering and exiting a patient collected from the temperature sensor over a discrete period of time. The discrete time period can be less than 5 minutes, less than 4 minutes, less than 3 minutes, less than 2 minutes or less than 1 minute.

In some embodiments, processing the data can further include fitting the temperature data to an oscillating wave pattern.

In some embodiments, the temperature sensor can include a thermistor.

In some embodiments, the method can further include signaling an anomaly in the data collected from the temperature sensor.

In some embodiments, a respiratory arrest/death event can include a cardiac or pulmonary event. In particular, the event can include cardiac arrest or respiratory arrest. In some circumstances, the event can include dysrhythmia, for example, cardiac dysrhythmia. In some circumstances, the event can include the patient becoming hypoxic, leading to respiratory arrest/death. In some circumstances, the event can include the inability to breathe, dysynchronous breathing, and/or loss of central stimulation to breathe. A respiratory arrest/death event can include cessation of breathing, hypoventilation, or a progressive hypoventilatory state.

In some embodiments, the continuous period during patient hospitalization can be at least two hours, at least four hours, at least eight hours, at least twelve hours, at least twenty four hours or at least forty eight hours.

In another aspect, a system for monitoring patients for a respiratory arrest/death event can include a temperature sensor, in fluid communication with air entering and exiting a patient, where the patient has been determined to be at risk of undergoing a respiratory arrest/death event, and a processor for processing data on a temperature of the air entering and exiting a patient collected from the temperature sensor over a continuous period during patient hospitalization.

In another aspect, a system for monitoring patients can include a temperature sensor, in fluid communication with air entering and exiting a patient, where the patient has been admitted to a hospital, and a processor for processing data on a temperature of the air entering and exiting a patient collected from the temperature sensor over a discrete period of time. The discrete time period can be less than 5 minutes, less than 4 minutes, less than 3 minutes, less than 2 minutes, less than 1 minute, less than 30 seconds, less than 15 seconds or less than 10 seconds.

In another aspect, a system for monitoring patients in an emergency vehicle can include a temperature sensor, in fluid communication with air entering and exiting a patient, where the patient is being transported by an emergency vehicle, and a processor for processing data on a temperature of the air entering and exiting a patient. The processor can process data on a temperature of the air entering and exiting a patient collected from the temperature sensor over a discrete period of time. The discrete time period can be less than 5 minutes, less than 4 minutes, less than 3 minutes, less than 2 minutes, less than 1 minute, less than 30 seconds, less than 15 seconds or less than 10 seconds.

In another aspect, a system for determining a respiratory rate of a patient can include a temperature sensor, in fluid communication with air entering and exiting a patient, and a processor for processing data on a temperature of the air entering and exiting a patient collected from the temperature sensor over a discrete period of time. The discrete time period can be less than 5 minutes, less than 4 minutes, less than 3 minutes, less than 2 minutes, less than 1 minute, less than 30 seconds, less than 15 seconds or less than 10 seconds.

In some embodiments, the temperature sensor can include a thermistor.

In some embodiments, the system can further include a signal generator, which can be activated by an anomaly in the data collected from the temperature sensor.

In some embodiments, the respiratory arrest/death event can be a cardiac or pulmonary event. In particular, the event can be cardiac arrest or respiratory arrest. In some circumstances, the event can include dysrhythmia.

In some embodiments, the continuous period during patient hospitalization can be at least two hours, at least four hours, at least eight hours, at least twelve hours, at least twenty four hours or at least forty eight hours.

Other embodiments are within the claims.

### BRIEF DESCRIPTION OF THE DRAWINGS

FIG. 1 is a diagram depicting a system for monitoring a patient for sudden respiratory death.

### DETAILED DESCRIPTION

When a patient arrives at the hospital, the patient is typically evaluated and assigned a risk level for undergoing a cardiopulmonary arrest/death event. The risk level of the patient can change over the period of time during which the patient is in the hospital. As the risk level of the patient changes, the care the patient requires can also change. As a result, the patient may need to be transferred to a different physician, department, actual location and/or virtual location. For example, a high risk patient may be required to be in a first location. When the patient's risk decreases, the patient can be transferred to a second location. As another example, a low risk patient may be required to be in a first location. When the patient's risk increases, the patient can be transferred to a second location. Alternatively, the patient can have a virtual location change, meaning the patient can remain in the same actual location but the level of monitoring and/or care can be decreased or increased. For example, as a patient's risk increases, the level of monitoring can increase, including monitoring using the system described herein.

Current issues with this system include backlogs within locations that occur when patients are ready to “stepdown” to a lower risk level location, but the lower risk level location does not have enough space. Additionally, this system can require the multiple patient transfers that can occur when a relatively large number of patients need access to a limited supply of equipment that exists at different locations.

To solve this problem, a flexible monitoring system has been implemented, where the vital signs, including cardiac rhythm or pulse oximetry, can be monitored from a remote location by monitoring technicians. The flex monitoring system can allow for the monitoring of any patient, in any bed, without the patient changing bed location or department.

Patients at a risk of undergoing a sudden respiratory death risk have traditionally been monitored by electrocardiography (“ECG”). ECG monitoring can capture tachy dysrhythmias that can be a cause of preventable sudden respiratory death. Historically, it was believed that this was the most cost effective way to detect preventable sudden respiratory death of patients early. However, some patients experience brady dysrhythmias in the ten minutes prior to the calling of a code blue. Brady dysrhythmias are associated with hypoxia, which indicates that respiratory arrest, and not cardiac arrest, may be resulting in a respiratory arrest/death event in these patients. ECG monitoring may not detect the respiratory arrest early enough to allow for successful resuscitation. Respiratory monitoring technology exists. One approach currently available for monitoring respiratory arrest is pulse oximetry. Additionally, respiratory rate monitors, expired and/or transcutaneous CO₂ monitoring, and air flow recording devices have also been used. Current respiratory rate and CO₂ monitoring devices, however, are not always reliable nor do they produce reproducible data in the awake and active patient. CO₂ monitoring can also be expensive and difficult to calibrate.

Therefore, a need exists for developing a reliable and reproducible system for monitoring an at-risk patient for respiratory arrest.

In one embodiment, a system can be designed to measure respiratory rates and possibly minute ventilation on a continuous basis. Utilizing temperature sensor technology, for example a thermistor, the system can measure temperature changes during the cyclic air movement of each breath. The temperature sensor can be placed in or near an air passage, such as a nostril or mouth, for example, within the orifice of a nostril. The system can work with oxygen flowing through a nasal cannula or a high flow face mask. The system can be utilized with respiratory rates up to the mid 30's with no loss in sensitivity. The system can also work with only mouth breathing.

In a preferred embodiment, the system can be deployed by placing the device into the nasal opening and having it secured either with an adhesive tape or some type of strapping system. The system can also be clipped to nasal prongs or some other securing device. A connecting wire can be threaded behind the ipsilateral ear and then plugged into a transmitting device. In some embodiments, the transmitting device can be directly attached to the temperature sensor, for example, near the nose. In other embodiments, the transmitting device can wirelessly transmit temperature data from the temperature sensor. The transmitting device can be the same size and have the same characteristics of the ECG transmitter used with a traditional wireless ECG telemetry system.

The system can have computer generated alarms to notify care givers of reductions in respiratory rates and potentially reductions in minute ventilation. The change in minute volume can be acquired from the reductions in amplitude of the temperature wave form as the patient reduces their breath-to-breath tidal volume.

In one aspect, a system for monitoring patients for a respiratory arrest/death event can include a temperature sensor in fluid communication with air entering and exiting a patient, where the patient has been determined to be at risk of undergoing a respiratory arrest/death event, and a processor for processing data on a temperature of the air entering and exiting a patient collected from the temperature sensor over a continuous period during patient hospitalization.

In another aspect, a system for monitoring patients can include a temperature sensor, in fluid communication with air entering and exiting a patient, where the patient has been admitted to a hospital, and a processor for processing data on a temperature of the air entering and exiting a patient collected from the temperature sensor over a discrete period of time. The discrete time period can be less than 5 minutes, less than 4 minutes, less than 3 minutes, less than 2 minutes, less than 1 minute, less than 30 seconds, less than 15 seconds or less than 10 seconds.

In another aspect, a system for monitoring patients in an emergency vehicle can include a temperature sensor, in fluid communication with air entering and exiting a patient, where the patient is being transported by an emergency vehicle, and a processor for processing data on a temperature of the air entering and exiting a patient. The processor can process data on a temperature of the air entering and exiting a patient collected from the temperature sensor over a discrete period of time. The discrete time period can be less than 5 minutes, less than 4 minutes, less than 3 minutes, less than 2 minutes, less than 1 minute, less than 30 seconds, less than 15 seconds or less than 10 seconds.

In another aspect, a system for determining a respiratory rate of a patient can include a temperature sensor, in fluid communication with air entering and exiting a patient, and a processor for processing data on a temperature of the air entering and exiting a patient collected from the temperature sensor over a discrete period of time. The discrete time period can be less than 5 minutes, less than 4 minutes, less than 3 minutes, less than 2 minutes, less than 1 minute, less than 30 seconds, less than 15 seconds or less than 10 seconds.

In another aspect, a method for monitoring patients for a respiratory arrest/death event can include selecting a patient that is at risk of undergoing a respiratory arrest/death event, attaching a temperature sensor, to the patient such that the temperature sensor is in fluid communication with air entering and exiting the patient, and processing data on a temperature of the air entering and exiting the patient collected from the temperature sensor over a continuous period during patient hospitalization.

In another aspect, a method for detecting respiratory changes can include attaching a temperature sensor to a patient such that the temperature sensor is in fluid communication with air entering and exiting the patient, processing data on a temperature of the air entering and exiting the patient collected from the temperature sensor over a continuous period during patient hospitalization. The method can further include transmitting a signal through a wireless device for remote monitoring.

In one aspect, a method for monitoring patients can include selecting a patient who has been admitted to a hospital, attaching a temperature sensor to the patient during the collection of vital sign information, such that the temperature sensor is in fluid communication with air entering and exiting the patient for a discrete time period, and processing data on a temperature of the air entering and exiting the patient collected from the temperature sensor. The discrete time period can be less than 5 minutes, less than 4 minutes, less than 3 minutes, less than 2 minutes or less than 1 minute.

In one aspect, a method for monitoring patients can include selecting a patient who is being transported by an emergency vehicle, attaching a temperature sensor to the patient during the collection of vital sign information, such that the temperature sensor is in fluid communication with air entering and exiting the patient, and processing data on a temperature of the air entering and exiting the patient collected from the temperature sensor. The processor can process data on a temperature of the air entering and exiting a patient collected from the temperature sensor over a discrete period of time. The discrete time period can be less than 5 minutes, less than 4 minutes, less than 3 minutes, less than 2 minutes or less than 1 minute.

In another aspect, a method for determining a respiratory rate of a patient can include attaching a temperature sensor to the patient, such that the temperature sensor is in fluid communication with air entering and exiting the patient, and processing data on a temperature of the air entering and exiting the patient collected from the temperature sensor. The processor can process data on a temperature of the air entering and exiting a patient collected from the temperature sensor over a discrete period of time. The discrete time period can be less than 5 minutes, less than 4 minutes, less than 3 minutes, less than 2 minutes or less than 1 minute.

In some embodiments, processing the data can further include fitting the temperature data to an oscillating wave pattern.

The temperature sensor can include a thermistor.

The patient can be an animal, such as a mammal, for example, a human, dog, cat, horse, cow, rat, rabbit or mouse. The patient can be a child or an adult. The patient can be at least 2 years old, at least 5 years old, at least 10 years old, at least 12 years old, at least 15 years old or at least 18 years old.

The temperature sensor can be in fluid communication with air entering and exiting a patient. The temperature sensor can be located at a point in the respiratory passageway, including the mouth, the throat, trachea or the nose. The system can work with breath entering through the nostril, the mouth or both. In some embodiments, the temperature sensor can include channels through which the air can flow. In other embodiments, the temperature sensor can include extensions that protrude into the respiratory passageway. The temperature sensor can be located on the end of extensions. A temperature sensor can be located, for example, within a channel or on the end of an extension.

The temperature sensor can be connected to a temperature output for transmitting data to a processor. The data can be transmitted directly to a processor from the temperature output. The data can also be communicated indirectly to the processor. For example, the temperature output can be connected to a transmitter that transmits data to a processor at a remote location. The data transmission can be continuous, in real-time or at a scheduled delay. Alternatively, data can be transmitted in a batch, where data collected over multiple time points is transmitted in at one time. Data can be transmitted in one or more batches.

The temperature sensor may be combined with another monitoring device, for example, an oxygen sensor, a carbon dioxide sensor or pulse rate monitor. The temperature sensor can also be combined with a device for providing a gas to the patient, for example, a cannula delivering oxygen.

In some embodiments, the temperature sensor can be held in position by an attachment device. The attachment device can be an adhesive, for example tape, a strap, for example, a strap that encircles the patient's head, a hook, or a clamp.

A patient's risk can be an increased probability of undergoing a respiratory arrest/death event. In other words, the greater the probability that the patient will undergo a respiratory arrest/death event, the greater the patient's risk of adverse change in health.

The risk can be determined by a physician or other health care provider. The determination can include analysis of vital signs, signs observed and/or symptoms described. Vital signs analyzed can include pulse, blood pressure, respiratory rate and temperature. Signs observed can include level of consciousness; skin characteristics, for example, color, turgor and temperature; the ability to move, for example, the ability to lay back; the ability to communicate, for example, speaking in full sentences; tenderness or discomfort, including in the abdomen, chest or other body location. Symptoms can include shortness of breath, tight feeling or pressure in the chest, chest pain, jaw pain, back pain, stomach pain, nausea, vomiting, sweating, vertigo, anxiety, lightheadedness or dizziness.

A respiratory arrest/death event can include a cardiac or pulmonary event. In particular, the event can include cardiac arrest or respiratory arrest. In some circumstances, the event can include dysrhythmia, for example, cardiac dysrhythmia. In some circumstances, the event can include the patient becoming hypoxic. In some circumstances, the event can include the inability to breathe, dysynchronous breathing, and/or loss of central stimulation to breathe. A respiratory arrest/death event can include cessation of breathing, hypoventilation, or a progressive hypoventilatory state.

A patient's risk, along with the availability of hospital resources, can be factors for selecting a patient. In addition, specific therapies may require monitoring; examples include intravenous or intrathecal analgesics and/or sedatives. Medications, for example, medications that are know to have potential serious/life threatening side effects or allergic reactions, that the patient has been administered can also be a factor. Administration of medication includes administration performed by the patient or by someone else.

A continuous period during patient hospitalization can be for at least two hours, at least four hours, at least eight hours, at least twelve hours, at least twenty four hours or at least forty eight hours.

In some embodiments, the system can further include a signal generator, which can be activated by an anomaly in the data collected from the temperature sensor. A signal generator can create a sensory notification signal, directly or indirectly, to a responsible person, for example a visual signal, an auditory signal or a tactile signal. A visual signal can include an alert on a computer screen, an email, a text message, a pager message, a light or a flashing light. An auditory signal can include a phone call, a voice message or an alarm. A tactile signal can include vibration. A signal generator can alert the patient, a person at the location of the patient, and/or a person at a remote location.

In some embodiments, the system can include a signal generator, which can provide a signal. The signal can be readout or representation of the data collected by the temperature sensor. For example, the signal generator can provide a numerical value, an average value, a chart, a graph, a picture, a graphic, a color, a light, a word or a phrase. The signal can change as new data is transmitted to the signal generator from the temperature sensor.

A processor can process data on a temperature of air entering and exiting the patient collected from the temperature sensor. The data can include the temperature of the air at different time points. As air enters and exits the patient, the temperature over the temperature sensor can fluctuate as the air passes over the temperature sensor. The processor can analyze the data to determine a breathing pattern of the patient. For example, each time the patient breaths in, the temperature at a point within the respiratory passageway can decrease. Then, as the patient exhales, the temperature can increase and approach body temperature. Consequently, an oscillating pattern of increasing and decreasing temperatures can result from normal patient breathing. Temperatures detected by the temperature sensor can be influenced by the location of the temperature sensor, the rate of patient breathing, the air volume of patient breath, the patient's body temperature, the room temperature and/or the velocity of the air. The pattern can be identified by monitoring time passing between inflection points as temperature changes with time. If, however, the patient stops breathing, the oscillating pattern will change. The processor can detect an anomaly in the data collected from the temperature sensor.

In a preferred embodiment, the anomaly is the absence of an inflection point after a designated period of time passed from an initial inflection point. The designated period of time can be 10 seconds, 15 seconds, 20 seconds, 30 seconds, 40 seconds, 50 seconds, 60 seconds, 75 seconds, 90 seconds, 105 seconds, 120 seconds, or longer. The designated period of time can be selected based on the age or physical condition of the patient, the time of day or other known co-morbid conditions.

The designated period of time can be altered for individual patients. Factors that can be used in determining the designated period of time can include, but are not limited to, the patient's preexisting pattern of breathing, pulse oximetry data collected from the patient, ECG data collected from the patient, known patient conditions, known patient medications, lung function, age, weight, height, and sex. The location of the temperature sensor can also be a factor.

In some embodiments, the anomaly can be a significant change in the temperature range covered in one breath cycle, i.e. one inhalation and one exhalation. For example, a significant change in the temperature range can be a decrease of at least 20%, at least 30%, at least 40%, at least 50%, at least 60%, at least 70%, at least 80% or at least 90%. In some embodiments, the anomaly can be a significant change in the maximum or minimum temperature recorded in a breath cycle. A significant change in the maximum or minimum temperature can be a change of at least 2° F., at least 3° F., at least 4° F., at least 5° F., at least 6° F., at least 7° F., at least 8° F. or at least 10° F.

The change in temperature range that is considered an anomaly can be altered for individual patients. Factors that can be used in determining the change in temperature range that is considered an anomaly can include, but are not limited to, the patient's preexisting average temperature range, the maximum temperature recorded from the patient, the minimum temperature recorded from the patient, pulse oximetry data collected from the patient, ECG data collected from the patient, known patient conditions, known patient medications, lung function, age, weight, height, and sex. The location of the temperature sensor can also be a factor.

The change in the maximum or minimum temperature recorded in a breath cycle that is considered an anomaly can be altered for individual patients. Factors that can be used in determining the change in the maximum or minimum temperature that is considered an anomaly can include, but are not limited to, the patient's preexisting average temperature range, the maximum temperature recorded from the patient, the minimum temperature recorded from the patient, pulse oximetry data collected from the patient, ECG data collected from the patient, known patient conditions, known patient medications, lung function, age, weight, height, and sex. The location of the temperature sensor can also be a factor.

In some embodiments, the data collected from a temperature sensor can be transmitted to processor. The temperature sensor can be attached or connected to the processor. The connection can be via a wire or cord, or the connection can be a wireless connection.

In some embodiments, a system can include one or more portable components. For example, the system can include a portable data processor or a portable signal generator.

In an embodiment, a system can include a temperature sensor in fluid communication with air entering and exiting a patient, where the patient has been determined to be at risk of undergoing a respiratory arrest/death event, and a portable processor for processing data on a temperature of the air entering and exiting a patient collected from the temperature sensor over a continuous period of patient hospitalization. The data can be transmitted from the temperature sensor to the processor via a wireless connection. The system can further include a portable signal generator, which can be connected to the data processor via a wire or cord, or via a wireless connection.

In another embodiment, a method for monitoring patients can include selecting a patient that is at risk of undergoing a respiratory arrest/death event, attaching a temperature sensor to the patient such that the temperature sensor is in fluid communication with air entering and exiting the patient, transmitting data collected from the temperature sensor to a portable processor, and processing data on a temperature of the air entering and exiting the patient collected from the temperature sensor over a continuous period during patient hospitalization. Transmitting the data collected from the temperature sensor to a processor can be done via a wireless connection. The method can further include generating a signal from a portable signal generator. The signal generator can be connected to the data processor via a wire or cord, or via a wireless connection

In some embodiments, the data processor and/or signal generator can be connected to devices in addition to the temperature sensor. For example, the data processor may also be connected to a device measuring heart rate, such that the data processor can transmit data to the signal generator including both heart rate and respiratory rate information.

In some embodiments, a portable data processor can transmit data to a signal generator and a remote data storage or remote data processor. In this way, a medical professional can receive the data from the data processor and the data from the data processor can be stored, for example, in a medical record system.

In some embodiments, a signal generator can include a printer for printing the representation of the data received from the data processor.

A system or a method, as disclosed above, can be used to monitor patients, for example, while vital signs are being acquired or monitored. A person who is a medical professional, for example, a doctor, physician's assistant, nurse, nurse practitioner, medical technologist or other hospital personnel, can place the temperature sensor in fluid communication with air entering and exiting a patient. The temperature sensor can collect data, continuously or at multiple points in time. The data collected from the temperature sensor can be transmitted from the temperature sensor to the processor, which can then process the data and provide a processed data to a signal generator. The temperature sensor, the data processor and the signal generator can be separate or combined in one device that includes at least two of the temperature sensor, the data processor and the signal generator. The temperature sensor, the data processor and the signal generator can transmit data between each other via a wire or cord, or via a wireless connection.

For example, in some embodiments, the processor can be part of a monitoring device with the temperature sensor. The monitoring device can transmit signal to a signal generator via a wire or cord, or via a wireless connection. The signal generator can be part of a portable device, which can receive data from the temperature sensor and data processor.

As another example, in some embodiments, the data processor and the signal generator may be part of a portable device, and the temperature sensor can transmit signal to a signal generator via a wire or cord, or via a wireless connection.

The time from data collection with the temperature sensor to when a signal is generated by the signal generator can be less than less than 2 minutes, less than 1 minute, less than 30 seconds, less than 10 seconds, less than 5 seconds or substantially in real-time.

The medical professional can wait for the data to be collected, processed and a signal to be generated. Alternatively, the medical professional can perform additional tasks while waiting for the data to be collected, processed and a signal to be generated. For example, the medical professional may monitor the patient's vital signs, interview the patient or review the patient's medical chart. A medical professional may view the data remotely once it is collected and transmitted to a data repository.

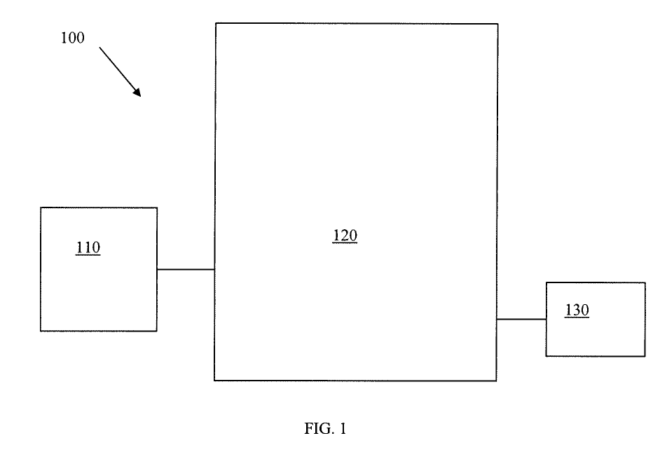

Referring to FIG. 1, a system for monitoring patients for a respiratory arrest/death event 100 includes a temperature sensor 110, a processor 120 for processing data on a temperature of the air entering and exiting a patient collected from the temperature sensor, and a signal generator 130. Portions of the system can be reusable. The temperature sensor can include a thermistor.

Temperature sensor 110 can be a device for measuring the temperature or temperature changes. More specifically, it can be a filamentous device, which may be partially or completely encased in a protective cover. The filamentous device can be 1 to 2 mm in diameter of various lengths.

System 100 can include one or more input devices, one or more output devices, a computer-readable medium, and a computer memory device. The processor 120 may be implemented using any computer processing device, such as, a general-purpose microprocessor or an application-specific integrated circuit (ASIC). Processor 120 can be integrated with input/output (I/O) devices to provide a mechanism to receive sensor data and/or input data and to provide a mechanism to display or otherwise output queries and results to a service technician. The input device may include, for example, one or more of the following: a mouse, a keyboard, a touch-screen display, a button, a sensor, and a counter.

An output device may be implemented using any output technology, including a liquid crystal display (LCD), a television, a printer, or a light emitting diode (LED). The system can include a computer-readable medium provides a mechanism for storing programs and data either on a fixed or removable medium. The computer-readable medium may be implemented using a conventional computer hard drive, or other removable medium. Finally, the system uses computer memory device, such as a random access memory (RAM), to assist in operating the demonstration system.

The signal generator 130 creates a visual signal or an auditory signal or sends the results of the processing to a system or individual monitoring the patient. The signal generator can include a user interface system employing one or more of the following sub-modules: image display module, text display module, video display module, or audio module, or a combination thereof. The image display module may be used to display images in various formats, for example, joint photographic experts group (JPEG) format, tagged image file format (TIFF), graphics interchange format (GIF), portable document format (PDF) or bitmap. The text display module may be used to display text messages, help messages, or other information to medical staff. In some implementations, the text display module can support the hypertext markup language (HTML) format such that displayed text may include hyperlinks to additional information, images, or formatted text. The video display module can provide a visual image. Finally, the audio module can provide a mechanism to produce a sound. The signal can be sent to local devices or transmitted to a remote location through a wired or wireless connection. It can be displayed on a PDA, beeper, local or remote central station, cell phone, or another electronic device configured to receive electronic/digital data.

Other implementations are within the scope of the following claims.

What is claimed is:

1. A method for monitoring patients for a respiratory arrest/death event comprising:
selecting a patient that is at risk of undergoing a respiratory arrest/death event;
attaching a temperature sensor to the patient such that the temperature sensor is in fluid communication with air entering and exiting the patient; and
processing data on a temperature of the air entering and exiting the patient collected from the temperature sensor over a continuous period during patient hospitalization.

2. The method of claim 1, further comprising signaling an anomaly in the data collected from the temperature sensor.

3. The method of claim 1, wherein the respiratory arrest/death event includes a cardiac or pulmonary event.

4. The method of claim 1, wherein the continuous period during patient hospitalization is at least two hours.

5. The method of claim 1, wherein the continuous period during patient hospitalization is at least four hours.

6. The method of claim 1, wherein the temperature sensor includes a thermistor.

7. A system for monitoring patients for a respiratory arrest/death event comprising:
a temperature sensor in fluid communication with air entering and exiting a patient, wherein the patient has been determined to be at risk of undergoing a respiratory arrest/death event; and
a processor for processing data on a temperature of the air entering and exiting a patient collected from the temperature sensor over a continuous period of patient hospitalization.

8. The system of claim 7, further comprising a signal generator, which is activated by an anomaly in the data collected from the temperature sensor.

9. The system of claim 7, wherein the respiratory arrest/death event includes a cardiac or pulmonary event.

10. The system of claim 7, wherein the continuous period of patient hospitalization is at least two.

11. The system of claim 7, wherein the continuous period of patient hospitalization is at least four hours.

12. The system of claim 7, wherein the temperature sensor includes a thermistor.

13. A method for monitoring patients for a respiratory arrest/death event comprising:
selecting a patient that is at risk of undergoing a respiratory arrest/death event;
attaching a temperature sensor to the patient such that the temperature sensor is in fluid communication with air entering and exiting the patient; and
processing data on a temperature of the air entering and exiting the patient collected from the temperature sensor over a continuous period during patient hospitalization.

14. A method for determining a respiratory rate of a patient comprising:
attaching a temperature sensor to the patient, such that the temperature sensor is in fluid communication with air entering and exiting the patient; and
processing data on a temperature of the air entering and exiting the patient collected from the temperature sensor over a discrete period of time.

15. The method of claim 14, wherein the discrete period of time is less than 1 minute.
